(19) United States
(12) Patent Application Publication
(10) Pub. No.: US 2004/0192594 A1
(43) Pub. Date: Sep. 30, 2004

(54) Modified neurotoxins as therapeutic agents for the treatment of diseases and methods of making

(76) Inventors: Paul Reid, Ft. Lauderdale, FL (US); Laurence Raymond, Ft. Lauderdale, FL (US)

Correspondence Address: Robert J. Van Der Wall, Wachovia Financial Center, Suite 5100, 200 South Biscayne Boulevard, Miami, FL, 33131-2310, US

(21) Appl. No.: 10/352,335

(22) Filed: Jan. 27, 2003

Related U.S. Application Data
(60) Provisional application No. 60/351,462, filed on Jan. 28, 2002.

(51) Int. Cl. A61K 38/16

(52) U.S. Cl. 514/12

(57) ABSTRACT

Disclosed is a method for treatment of neurological and viral diseases and especially to the treatment of heretofore intractable diseases such as Rabies, Myasthenia Gravis, HIV Dementia, Muscular Dystrophy, Multiple Sclerosis and Amyotrophic Lateral Sclerosis through modulation or blockade of the nicotinic acetylcholine receptor. Also disclosed is the treatment composition of matter and methods of making same. Treatment is based on the fact that certain modified alpha-neurotoxins have the ability to attach to or otherwise modulate the nicotinic acetylcholine receptor by blocking attachment or involvement with pathogenic organisms, viruses, or proteins with potentially deleterious functions. The modified alpha-neurotoxins may be derived from various venoms including certain genera of snakes and Conus snails and are prepared by detoxification of the purified neurotoxins or contained in whole venom. The native neurotoxin or venom may be detoxified by controlled oxygenation. A novel high temperature technique is also described. Alternatively, the specific neurotoxin may be generated through cloning or synthetic techniques with mutations or non-native amino acids substituted to reduce the affinity of the resulting neurotoxin for its receptor. The present composition may also be produced from any venom which acts, essentially, as a neurotoxin, as opposed to, essentially, a hematoxin. However, the composition must be derived from venoms which contains alpha-neurotoxins such as obtained from the genus Bungarus.

### CROSS REFERENCE TO RELATED APPLICATION

The present application continues from a provisional patent application serial No. 60/351,462 filed Jan. 28, 2002, and claims the filing date thereof as to the common subject matter.

### BACKGROUND OF THE INVENTION

1. Field of the Invention

The present invention relates to a class of proteins, a process of production thereof, and a method for treatment of neurological and viral diseases and especially to the treatment of heretofore intractable diseases such as Rabies, Myasthenia Gravis, HIV Dementia, Muscular Dystrophy, Multiple Sclerosis and Amyotrophic Lateral Sclerosis through modulation or blockade of the nicotinic acetylcholine receptor. The composition consists of modified anticholinergic neurotoxins which retain the ability to interact with their respective receptors.

2. Description of the Prior Art

Sanders et al. had commenced investigating the application of modified venoms to the treatment of ALS in 1953 having employed poliomyelitis infection in monkeys as a model. Others antiviral studies had reported inhibition of pseudorabies (a herpesvirus) and Semliki Forest virus (alpha-virus). See Sanders' U.S. Pat. Nos. 3,888,977, 4,126,676, and 4,162,303. Sanders justified the pursuit of this line of research through reference to the studies of Lamb and Hunter (1904) though it is believed that the original idea was postulated by Haast. See Haast U.S. Pat. Nos. 4,741,902 and 5,723,477. The studies of Lamb and Hunter (Lancet 1:20, 1904) showed by histopathologic experiments with primates killed by neurotoxic Indian cobra venom that essentially all of the motor nerve cells in the central nervous system were involved by this venom. A basis of Sanders' invention was the discovery that such neurotropic snake venom, in an essentially non-toxic state, also could reach that same broad spectrum of motor nerve cells and block or interfere with invading pathogenic bacteria, viruses or proteins with potentially deleterious functions. Thus, the snake venom used in producing the composition was a neurotoxic venom, i.e. causing death through neuromuscular blockade. As the dosages of venom required to block the nerve cell receptors would have been far more than sufficient to quickly kill the patient, it was imperative that the venom was detoxified. The detoxified but undenatured venom was referred to as being neurotropic. The venom was preferably detoxified in the mildest and most gentle manner. While various detoxification procedures were known then to the art, such as treatment with formaldehyde, fluorescein dyes, ultraviolet light, ozone or heat, it was preferred that gentle oxygenation at relatively low temperatures be practiced, although the particular detoxification procedure was not defined as critical. Sanders employed a modified Boquet detoxification procedure using hydrogen peroxide, outlined below. The acceptability of any particular detoxification procedure was tested by the classical Semliki Forest virus test, as taught by Sanders, U.S. Pat. No. 4,162,303.

From 1972 to 1974, 113 patients were treated for ALS with the crude venom extract without reports of toxicity problems or other adverse reactions (Sanders, M. and Fellowes, 1975). The objective of the treatment was an attempt to decelerate, stabilize or possibly reverse the progression of the disease. The response in patients after an average treatment period of 14 months was reported. In the evaluation of patient survival it was necessary to consider the severity of the disease at the time treatment was initiated. Those with severe disease did not respond well to treatment. Those with lesser grades of involvement survived beyond 12 years. Overall a 68% survival rate was estimated. An IND (BB1073) from the Food and Drug Administration was in effect from 1972 to 1987. During that period, a product derived from oxidatively detoxified whole venoms (cobra and krait) was employed as a therapeutic agent in over 1,100 patients with Amyotrophic Lateral Sclerosis (ALS) with the longest treated patients receiving treatment for over 12 years. The venom complex contained many potentially active components though the emphasis of research efforts have focused on the neurotoxic fraction. The treatment group received 0.1-2 ml of oxidized whole snake venom at a concentration of 10 g/L (10 mg/ml) every other day. In the neurotoxic fraction, cobratoxin represented from 15-20% of the venoms excluding a number of other neurotoxin homologues (cobrotoxin 5%, muscarinic toxins <0.1%, alpha-bungarotoxin 0.01% and kappa-bungarotoxin <0.001%).

Several other investigators conducted placebo controlled studies in patients with ALS with Sanders' modified venom preparation employing the same dosages. While the published reports did not confirm efficacy no safety concerns were raised. In these combined studies a total of 112 patients were involved (Tyler, 1979, Rivera et al., 1979). However, if these published results are closely scrutinized issues are raised over the failure of the medication are focused upon the duration (6 months), clinical endpoints employed in those investigations in addition to confusing reports of efficacy. In fact, subsequent to the published report of Rivera et al., Rivera acknowledged that some of the treated patients survived and remained stable. With revised clinical endpoints in place, Rivera also performed an open study in which he reported at a neurological meeting that 46% of the patients in this study were either stabilized or, in some cases, showed improvement. It is unknown what components of the venom were responsible for any benefits reported by Sanders. In patent issued to Haast, it was suggested that a combination of neurotoxins and an unknown component of viperid venom were required. (Sanders did not employ a viperid venom component). Haast employed native, unmodified venom fractions the administration of which was reported to cause quite extensive pain for 1-2 days post administration resulting often in short therapeutic periods even if the effects were quite dramatic.

The production of drug product by Dr. M. Sanders was achieved using hydrogen peroxide as the oxidizing agent in addition to other components giving the recipe he employed for over 30 years (Sanders et al., 1975, 1978). This method was patented and published by Sanders on several occasions with the last patent expiring in 1994. Furthermore, several techniques have been developed for modifying neurotoxins to yield a potentially therapeutic product though many have not be reduced to practice. These have included hydrogen peroxide, ozone, performic acid, iodoacetamide and iodoacetic acid. Some of these procedures have been published and others patented. Obviously some procedures are easier than others to utilize and the focus for commercial production has been on the simpler methods.

Other references of interest include two patents, Haast, U.S. Pat. No. 4,341,762; Cosford, et al., U.S. Pat. No. 5,585,388, which claims compounds as modulators of acetylcholine receptors. Literature references of interest are: Atassi M Z, Manshouri T. and Yokoi T., FEBS Lett 1988 Feb. 15;228(2):295-300; Bracci, L., Antoni, G., Cusi, M., Lozzi, L., Niccolai, N. Et.al.; Mol. Immunol. 25:881 888 (1988); Brenner, T., Timore, Y., Wirguin, I., Abramsky, O. and Steinitz, M., J. Neuroimmunol., (1989), 24, 217-22; Burrage T. G., Tignor G. H., and Smith A. L.; Virus Res 2: 273-289 (1985); Carlson N. G., Bacchi A., Rogers S. W., Gahring L. C., J. Neurobiol 1998 April;35(1):29-36; Chuang L. Y., Lin S. R., Chang S. F. and Chang C. C. Toxicon 27:211-219 (1989); Dargent B, Arsac C, Tricaud N, Couraud F., Neuroscience 1996 July;73(1):209-16; Dierks R. E., Murphy F. A., and Harrison A. K. Am. J. Pathol. 54: 251-274 (1969); Duggan, D. B., Mackwoth-Young, C., Kari-Lefvert, A., Andre-Schwartz, J., Mudd, D., McAdam K. amd Schwartz, R., Clin. Immunol. Immunopath. (1988) 49, 327-40; Hinmann C. L., Stevens-Truss R., Schwarz C., Hudson R. A. Immunopharmacol Immunotoxicol. (1999) August;21(3):483-506., Hudson R A, Montgomery I N and Rauch H C. Mol Immunol. (1983) Feb.;20(2):229-32; Kase R., Kitagawa H., Hayashi K., Tanoue K. And Inagaki F.; FEDS Lett 254:106-110 (1989); Lamb, G and Hunter, W. K, The Lancet, 1: 20-22; Lentz, T. C., Hawrot, E. and Wilson, M, Proteins: structure, function and genetics. (1987) 2; 298-307; Lentz, T. L., Burrage, T. G., Smith A. L., Crick, J., Tignor G. H.; Science 215:182-184 (1982); Lentz T. L., Hawrot E. And Wilson P. T.; Proteins: Structure, Function and Genetics 2:298-307 (1987); Lentz T. L., and Wilson P. T.; Int. Rev. Neurobiol. 29:117-160 (1988a); Lentz T. L., Hawrot E., Donnelly-Roberts D. And Wilson P. T.; Psychological, Neuropsyshiatric and Substance Abuse aspects if AIDS; edit by T. P. Bridge et.al., Raven Press, NY, (1988b); pp 57-71; Lentz,T; Biochem 30:10949-10957 (1991); Marx, A., Kirckner, T., Hoppe, F., O'Connor, R., Schalke, B., Tzartos, S. and Muller-Hermelink, H. K., Amer. J. Path, (1989) 134, No.4, 865-75; Miller, K., Miller, G. G., Sanders, M. And Fellowes, O. N., Biophys et Biophysica Acta 496:192-196) (1977); Neri P., Bracci L., Rustiel M., and Santucci A.; Arch Vitol 114:265-269 (1990); Patterson, B., Flener, Z., Yogev, R. and Kabat, W., Apr. 7, (2000), Keystone Conference, Colorado; Pillet L., Charpentier I., Leonetti M, Menez A. Biochim Biophys Acta (1992) Apr. 4;1138(4):282-9; Renshaw G M, Dyson S E. Neuroreport 1995 Jan. 26;6(2):284-8; Robinson, D. And McGee, R., Mol. Pharm. 27:409-417 (1985); Sanders, M., Soret, M. G. and Akin, B. A.; Ann. N. Y. Acad. Sci. 53: 1-12 (1953); Sanders, M., Soret, G., and Akin, B. A.; J. Path. Bacteriol. 68:267-271 (1954); Sanders M. And Fellows O.; Cancer Cytology 15:34-40(1975) and in Excerpta Medica International; Congress Series No. 334 containing abstracts of papers presented at the III International Congress of Muscle Diseases, Newcastle on Tyne, September 1974; Sanders M., Fellowes O. N. and Lenox A. C.; In: Toxins: Animal, Plant and Microbial, Proceedings of the fifth international symposium; P. Rosenberg, editor, Pergamon Press, New York 1978, p. 481; Saroff D., Delfs J., Kuznetsov D., Geula C., Neuroreport 2000 Apr. 7;11(5):1117-21; Tseng, L. F., Chiu, T. H., and Lee, C. Y.; Tox. Appl. Pharmac. 12:526-535 (1968); Tsiang H., de la Porte S., Ambroise D. J., Derer M. And Koenig J.; J. Neuropathol. Exp. Neurol. 45: 28-42; Tu A. T.; Ann. Rev. Biochem. 42:235-258(1973); Umemura, K., Gemba, T., Mizuno, A. and Nakashima, M, Stroke. 1996;27:1624-1628; Urushitani M, Nakamizo T, Inoue R, Sawada H, Kihara T, Honda K, Akaike A, Shimohama S. J.; Neurosci Res 2001 Mar. 1;63(5):377-87; and Xu L., Villain M., Galin F. S, Araga S, Blalock J E., Cell Immunol. (2001) Mar. 15;208(2):107-14.

### SUMMARY OF THE INVENTION

It is an object of the invention to provide a composition and method for treating viral and progressive degenerative diseases of the nervous system which involve the function of the nicotinic acetylcholine receptor, such as rabies, HIV dementia, amyotrophic lateral sclerosis, multiple sclerosis, muscular dystrophy, myasthenia gravis and the like.

It is a further object of the invention to provide a composition and therapy for the treatment of diseases of the aforementioned type, which composition and therapy are safe, effective and may be administered over long periods of time.

Another object of the invention to provide a method of manufacture of the composition of the present invention.

Other objects will be apparent to those skilled in the art from the following disclosures and and appended claims.

The present invention accomplishes the above-stated objectives, as well as others, as may be determined by a fair reading and interpretation of the entire specification.

Bearing in mind the foregoing, a principal aspect of the invention is that it has now been discovered that certain modified alpha-neurotoxins have the ability to attach to or otherwise modulate the nicotinic acetylcholine receptor by blocking attachment or involvement with pathogenic organisms, viruses, or proteins with potentially deleterious functions. The modified alpha-neurotoxins may be derived from various venoms including certain genera of snakes and Conus snails and are prepared by detoxification of the purified neurotoxins or contained in whole venom.

In accordance with another aspect of the invention, there is provided a method of drug production by modification of the procedure by Sanders, in which the native neurotoxin or venom is detoxified by controlled oxygenation, although any of the known detoxification procedures may be used with the exception of certain methods used to produce antivenom. A novel high temperature technique is also described. Alternatively, the specific neurotoxin may be generated through cloning or synthetic techniques with mutations or non-native amino acids substituted to reduce the affinity of the resulting neurotoxin for its receptor. The present composition may also be produced from any venom which acts, essentially, as a neurotoxin, as opposed to, essentially, a hematoxin. However, as will be more fully explained below, the composition must be derived from venoms which contains alpha-neurotoxins such as obtained from the genus Bungarus.

In accordance with a further aspect of the invention, there are provided alternative methods of drug production. These include heat treatment of alpha-neurotoxins. These novels methods of production give the option of generating proteins with subtle differences that have great importance to their application. Excessive exposure to heat is a mechanism that can be employed to investigate stability and heat-stress studies are commonly employed to assess the heat sensitivity of a protein and to simulate the passage of time.

### DETAILED DESCRIPTION OF THE INVENTION

As required, detailed embodiments of the present invention are disclosed herein; however, it is to be understood that the disclosed embodiments are merely exemplary of the invention which may be embodied in various forms. Therefore, specific functional details disclosed herein are not to be interpreted as limiting, but merely as a basis for the appended claims and as a representative basis for teaching one skilled in the art to variously employ the present invention in virtually any appropriate circumstance.

Anti-cholinergics are those drugs which antagonize the activity of acetylcholine and several have been used to treat the symptoms of a number of diseases. Acetylcholine is the major excitatory neuro-transmitter of the parasympathetic nervous system including the peripheral nervous system. This system can be divided into two systems; afferent and efferent. The afferent system transmits information (heat, cold, pain) to the CNS. The efferent transmits information from the CNS to muscles and glands. The efferent system can be further subdivided into the somatic and autonomic systems. The somatic system is under voluntary control. The autonomic system is responsible for involuntary control transmitting information to glands, smooth muscle and cardiac muscle. This is the system that current anticholinergic drugs have been designed to influence.

As antagonists of the acetylcholine receptor both alpha cobratoxin and alpha-bungarotoxin (alpha-neurotoxins) have found great utility as molecular probes in the study of neuro-muscular transmission and ion channel function. Eight different types of nicotinic acetylcholine receptors (NAchRs) have been identified with variable pharmacological profiles. A homologue, kappa-bungarotoxin, has a higher affinity for neuronal species of acetylcholine receptors. Other alpha-neurotoxins have been isolated from related species of snakes and fish-eating sea snails (Conus geographus, textilis, imperialis and striatus). Cobratoxin and alpha-bungarotoxin have highest affinity for nicotinic AchRs containing the alpha 1 and 7 subunits (for a review see Lucas, 1995). In the peripheral nervous system (PNS), the post synaptic response of nicotinic agonists is not blocked by alpha-bungarotoxin and alpha-bungarotoxin binding sites are located extra-synaptically and have a high permeability to calcium (Colquhoun and Patrick, 1997). The toxicity of these molecules is based upon their relative affinity for the receptor which far exceeds that of acetylcholine. Many studies (Miller et al., 1977, Hudson et al., 1983, Lentz et al., 1987, Donnelly-Roberts and Lentz, 1989, Chang et al., 1990, Fiordalisi et al., 1994) have demonstrated various methods for the chemical modification of cobratoxin, by oxidation with substances such as hydrogen peroxide, formalin and ozone, which result in an alteration in affinity for the acetylcholine receptor (AchR) and a concomitant loss in toxicity.

Cobratoxin and one of its homologues, bungarotoxin (BTX), target the nicotinic acetylcholine receptor (NAchR) in nerve and muscle tissue and functions by preventing depolarization of post-synaptic membranes through the regulation of ion channels. Cobratoxin (CTX) has a molecular weight of 7831 and is composed of 71 amino acids. It has no enzymatic activity (like botulinum, tetanus or ricin). It is toxic by virtue of its affinity for the acetylcholine receptor. Many such “neurotoxins” are very basic in nature, containing large numbers of such residues as lysine and arginine. Binding to the specific target is mediated primarily through electrostatic interactions of amide groups on the toxin to carboxyl groups on the receptor. High salt concentrations can interfere with such interactions. The structure of the protein has been determined by NMR and is composed mostly of antiparallel beta-sheets and random coil. These sheets form 3 loops, the central loop (loop 2) being essential for the protein's activity. Loop 2 contains the arginine-glycine motif, which is essential for the binding of alpha-neurotoxins. Shortened peptides (10 to 20mers) composed of residues from loop 2 can bind to the NAchR, though with lowered affinity, and prevent the activation of the receptors associated sodium channel. It should be noted that there are alpha-neurotoxin binding structures that are not acetylcholine receptors.

The administration of a highly toxic substance such as cobratoxin for therapeutic purposes is fraught with obvious difficulties, even when highly diluted. As a diluted substance, its potential effectiveness is reduced. As taught by Sanders, removal of the toxicity of cobratoxin can be achieved by exposure to heat, formalin, hydrogen peroxide, performic acid, ozone or other oxidizing/reducing agents. The result of exposure of cobratoxin to these agents is the modification of amino acids as well as the possible lysis of one or more disulfide bonds. Tu (1973) has demonstrated that the curaremimetic alpha neurotoxins of cobra and krait venoms lose their toxicity upon either oxidation or reduction and alkylation of the disulfide bonds which has been confirmed by Hudson et al (1983). Loss of toxicity can be determined by the intraperitoneal injection of excess levels of the modified cobratoxin into mice; in general a 1 mL volume containing 0.5-1 mg of modified cobratoxin is tested, which represents a minimum of a 400-fold reduction of toxicity. Alternatively, loss of toxicity can be evaluated by depression of binding of the modified neurotoxin to acetylcholine receptors (AchR) in vitro.

Modified cobra venom and cobratoxin in their oxidized (modified or non-toxic) forms have demonstrated antiviral activities. Native cobratoxin and formaldehyde-treated cobratoxin lack this activity (Miller et al., 1977). The mechanism by which this modified neurotoxin exerts this capacity is not clear as many viruses employ a variety cell surface receptors as portals for entry into the cell prior to replication.

### Relationship Between Viruses, Antibodies, Disease and the Acetylcholine Receptor

It has been proposed that the Rabies virus employs the nicotinic acetylcholine receptor (AchR) as its attachment point to gain entry into the cell. The high density of AchR at neuromuscular junctions could result in virus concentration, resulting in cross linking of receptors and internalization of the virus by muscle. Rabies virus glycoprotein and curare mimetic snake neurotoxin (as alpha bungarotoxin) share three-dimensional structures based upon primary structure amino acid sequence homologies, which result in binding to the AchR (Lentz et al., 1987, Bracci et al., 1988). Lentz et al. (1982) first reported binding in cultured chick myotubes could be inhibited by alpha bungarotoxin. Tsiang et al. (1986) reported a similar effect was observed in cultured rat myotubes. Subsequently a sequence homology between a segment of the rabies virus glycoprotein and snake venom curare mimetic neurotoxins was demonstrated (Lentz et al., 1982, Neri et al., 1990, See Table 1).

TABLE I

Amino Acid Sequence Homologies between Rabies,
HIV and Curaremimetic Toxins.

| | | |
|---|---|---|
| 1. | C D A F C S S R G K V | alpha Bungarotoxin (30-40) |
| 2. | C D I F T N S K G K R | Rabies virus (ERA & CVS |
| | | strains) (189-199) |
| 3. | C D A F C S I R G K R | alpha-cobratoxin (30-40), |
| | | Naja kaouthia |
| 4. | C D G F C S I R G K R | alpha-cobratoxin (30-40), |
| | | Naja naja naja |
| 5. | C D G F C S S R G K R | alpha-cobratoxin (30-40), |
| | | Naja naja |
| 6. | C D K F C S I R G P V | kappa Bungarotoxin (30-40) |
| 7. | F N I G T S I R G K V | HIV gp120 (164-174) |

As it has been proposed that the Rabies virus employs the nicotinic acetylcholine receptor (AchR) as its attachment point to gain entry into the cell it was chosen as a model system for this mechanism of viral inhibition and neurodegeneration. Previous rabies studies demonstrated that a tetradecapeptide corresponding to this specific region of the rabies virus resulted in the production of monoclonal antibodies (MoAb), some of which interacted with both the neurotoxin and rabies virus glycoprotein and could block binding of both alpha bungarotoxin and rabies virus to AchR from the electric organs of Torpedo maramorata. In addition, the ability of MoAb specific for Torpedo AchR alpha subunits to inhibit rabies virus binding at neuromuscular junctions was noted (Burrage et al., 1985) as well as the binding of radio-labeled rabies virus to Torpedo maramorata electric organs. The immunization of mice with rabies glycoprotein has been reported to result in auto-antibodies specific for AchR resulting in weight loss and death. Also, synthetic peptides corresponding to portions of the curaremimetic neurotoxin loop 2, specifically residues 25-44 of Ophiophagus hannah (king cobra; IC₅₀=5.7×10⁻⁶M {where IC₅₀ is the concentration of ligand resulting in a 50% reduction in binding of ¹²⁵I-alpha-Btx in the absence of competitor; [alpha Btx]=50 uL of 1 nm ¹²⁵I-alpha-Btx in an end volume of 400 uL consisting of 50 uL of competitor and 300 uL of solvent} (34) and the structurally similar segment of the CVS strain rabies glycoprotein (residues 173-203; IC₅₀=2.6×10⁻⁶M) had high affinities for Torpedo maramorta AchR which were comparable with those of d-tubocurarine (IC₅₀=3.4×10⁻⁶M) and suberyldicholine (IC₅₀=2.5×10⁻⁶M) (34). Thus loop 2 of curaremimetric snake neurotoxins and the rabies virus glycoprotein contain structurally similar segments which act as recognition sites for the AchR as well as having relatively high affinities for the AchR site (Lentz, 1991). Monoclonal antibodies (MoAb) produced in mice by immunization with HPLC fractionated peptide fragments from acid protease A digests of alpha bungarotoxin were found to neutralize the lethal activity of the toxin as well as to inhibit binding of the toxin to the nicotinic AchR. The epitope for which the MoAb is specific appears to involve residues 34-41 of BuTx (Chuang et al., 1989). A second group (Kase et al., 1999) has also developed a neutralizing MoAb which interacts with a BuTx fragment containing residues 34-38. Table II lists the IC₅₀ and relative affinity values with respect to the CVS rabies virus strain.

While rabies in humans has not as yet been treated with oxidized cobra venom or modified cobratoxin, there are several

TABLE II
Relative Affinity and IC50 Values Determined for Complete and
Specific Segments of Cholinergic Agents

| Agent | Residue | IC₅₀ (M) | Relative Affinity ^{(a)} |
|---|---|---|---|
| Antagonists | | | |
| alpha Bungarotoxin | entire | 8.4 × 10 − 9 | 30,952.0 |
| alpha cobratoxin | entire | 1.7 × 10 − 7 | 1,529.0 |
| d-Tubocurarine ^{(b)} | entire | 3.4 × 10 − 6 | 76.5 |
| Agonists | | | |
| Suderyldicholine | entire | 2.5 × 10 − 6 | 104.0 |
| Nicotine | entire | 1.4 × 10 − 3 | 0.19 |
| Carbamylcholinechloride | entire | 2.8 × 10 − 3 | 0.09 |
| Peptides | | | |
| CVS Rabies strain | 175-203 | 2.6 × 10 − 6 | 100.0 |
| King cobra | 25-44 | 5.7 × 10 − 6 | 45.60 |

reasons why they may be an effective mode of treatment—either by themselves or as an adjunct to the currently used immunization procedures. Such treatment may be advisable in cases if viral exposure occurs especially close to the brain—such as face, neck or shoulder administration of the virus by bites. A secondary application would utilize the modified cobratoxin as a vaccine to to generate antibodies that could inhibit the infectivity of rabies virus. This approach provides a composition that is both antiviral and immune stimulating.

There is also a notable sequence homology between alpha-cobratoxins and HIV gp-120 (Neri et al. 1990, Table 1) consisting of a stretch of 4-5 identical residues, which include the invariant amino acids (for the neurotoxin family) R37(arginine), G38 (glycine), K39 (Lysine) which are suggested to be involved in receptor binding. Such a sequence homology is of interest with respect to the ability of HIV to infect CD4 negative neuronal cells in culture (Harouse et al., 1989) as well as the inability of soluble CD4 (Clapham et al., 1989) and anti-CD4 antibody (Mebber et al., 1989) to block HIV binding to muscular and neuronal cells, suggesting infection by a route not mediated by CD4 and possibly through the AchR.

If the HIV can be prevented from entering cells then initial infection may be avoided and ongoing infection has the possibility of being controlled, perhaps even halted, by prevention of transfer of infection to uninfected cells. Thus fusion inhibitors have the potential to act as control/eradication agents and possibly prophylactically. Fusion inhibitors act by blocking the interaction of the HIV with the host cell surface. These sites are, CD4 and, most commonly, the CCR5 and CXCR4 co-receptors on the host cell (macrophages and T-lymphocytes) and gp-120 antigen on the HIV surface. If the fusion inhibitor binds at the appropriate site on either the host or HIV antigen surfaces, host-viral binding reactions will not occur and the virus will not gain entry to the host cell. Without ordered cell-HIV interaction, the virus cannot initiate genetic transfer and replication. This approach has validity based upon the finding that high levels of the native substances which interact with the CCR5 receptor, inhibit HIV infection of macrophages in vitro.

In general, the initial infection of an individual is caused by the HIV type that favors the CCR5 co-receptor on macrophages. This type of HIV-1 is termed M-tropic. For expansion of HIV within the infected host and as part of the expansion of the infection into the AID syndrome (AIDS) the virus changes its preferred co-receptor to CXCR4, which is functionally found on T lymphocytes. These HIV are designated as T-tropic. In both cases the CD4 receptor remains as the primary receptor for the HIV. The viral coat protein, gp120, attaches to the CD4 receptor during the initial stages of infection. The potential of neurotoxins as competitors for HIV receptors was proposed in 1990 by a research team in Italy. The hypothesis stemmed from the apparent homology between the viral coat protein (gp120) and the neurotoxin. HIV is also able to infect nerve cells in the absence of CD4 and a suggestion was made that the nerve cell receptor employed by HIV to enter the cell was the nicotinic acetylcholine receptor. The infection of nerve cells by HIV is assumed to lead to AIDS dementia. The major neurotoxins from cobras are specific for these types of receptor. Of note is the observation (Neri et al., 1990) that different members of the nicotinic acetylcholine receptor gene family are expressed in different regions of the mammalian CNS (Goldman et al., 1987). Neurologic dysfunction occurs in approximately 60% of AIDS patients (Ho et al., 1985) and sub acute encephalitis (AIDS encephalopathy or dementia complex) is a common neurologic problem, which seems to be specifically induced by HIV infection (Ho et al., 1985, Navia et al., 1986). Additionally, HIV has been isolated from brain, peripheral nerves and CSF of AIDS patients with sub acute encephalitis (Ho et al., 1985, Levy et al., 1985). Patterson et al. (2000) demonstrated that a detoxified cobra venom product could prevent the infection of thymus cells possibly through interaction with CD4 and chemokine receptors. However, HIV can infect CD4 negative cells and Bracci et al. (1992) showed that a peptide derived from gp120 could inhibit the binding of alpha-bungarotoxin to the nicotinic receptor.

Amyotrophic Lateral Sclerosis

Dissemination of Rabies to the spinal cord occurs via peripheral nerves by retrograde axonal transport followed by passage to the brain where infection is highly selective for certain neuronal populations and the resulting bulbar symptoms suggest also a component mimicking that of polio and ALS. Thus the blockade of rabies infection by modified alpha-neurotoxins suggest that they may also be effective in the treatment of neuro-degenerative disorders. This seems reasonable as it has also been reported that blockade of alpha-7 containing receptors, sensitive to cobratoxin and bungarotoxin, inhibited the release of glutamate, a potential trigger of cell apoptosis. Several studies have reported that people with ALS have a high level of glutamate circulating in the CNS. In stroke victims, the hypoxic state triggers a large outpouring of glutamate that kills the post-synaptic neuron (Unemura et al., 1996). Excitotoxic neuronal death mediated by N-methyl-D-aspartate (NMDA) glutamate receptors can contribute to the extended brain damage that often accompanies trauma or disease. Nicotine protection to NMDA was mediated through an alpha-bungarotoxin-sensitive receptor. When coapplied, neuroprotection to NMDA by nicotine was abolished but could be recovered with alpha-bungarotoxin. The study suggested that alpha-BTX-sensitive nicotinic neurotransmitter receptors confer neuroprotection through potentially antagonistic pathways (Carlson et al., 1997). It is interesting to note that alpha-7 receptors are expressed at low levels in the spinal chord so alpha-cobratoxin's effect may not be mediated there but further up the spinal chord or in the PNS. The cerebellum and other areas of the brain express high levels of toxin binding sites. Alpha-3 containing nicotinic receptors are more highly expressed in the spinal chord where the motor neurons are located. Kappa-bungarotoxin from the krait and other conus toxins are specific for alpha-3 containing receptors suggesting a combination of neurotoxins may ultimately prove to be the best approach. Kappa-bungarotoxin is present only in minute amounts (0.05%) in the venom so its contribution to the properties of Sanders 40:1 cobra:krait formula would, most likely be minimal where it is diluted to 0.0013%. Each 1 cc injection of a 4 L preparation would contain approximately 30-45 nanograms. It would argue for the formulation by Haast due to the higher krait:cobra neurotoxin ration estimated to give 10 nanograms/ml. While this is less than Sanders formula, it is unmodified and therefore more than 1000 times more potent. However, its' specificity and that of other alpha3 specific conotoxins would represent attractive therapeutic agents when modified in using the methods described herein.

The observation that alpha-Bungarotoxin (from the Krait, Bungarus multicintus), alpha-cobratoxin (from Naja kaouthia) and other curare-like drugs could arrest naturally occurring motor neuron death in embryonic chick spinal cord (Renshaw et al., 1993) encourage its inspection in animal models with motor neuron degeneration. This would be hampered by the fact the neurotoxins kill mice at very low doses (<2 mcg/mouse) but appropriate chemical detoxification of the toxins can overcome this impediment. Detoxification of neurotoxins, as described by Sanders, reduces the affinity for the receptor but it is not abolished. Renshaw did demonstrate that central nicotine-sensitive sites which bind alpha-bungarotoxin (BTX) were present at the beginning of the critical motor neuron death phase of neurogenesis and that they were accessible to exogenously administered toxin (Renshaw, 1994). Intramuscularly and intraperitoneally administered iodinated alpha-BTX reaches and binds to neuronal alpha-BTX-sensitive nicotinic cholinoceptors. Binding of alpha-BTX to these neuronal receptors and to those at the neuromuscular junction has now been shown to have a demonstrable effect on neuronal metabolism (Renshaw and Dyson, 1995). The decreased metabolic activity in spinal cord neurons as a result of toxin treatment may have an important role in the prevention of motoneuron apoptosis at a critical developmental phase. Tseng et al (1968) indicated that the CNS levels of mice and rabbits injected intravenously with CTX were very low. Pharmacokinetic studies performed in rabbits and humans by Miller et al. (1987) with modified CTX confirmed this observation. This may have two interpretations; CTX and BTX have different distribution properties in-vivo—a fact not observed before or access to the CNS is permissible during neurodegenerative disease. CTX is not toxic to cell lines in tissue culture at up to 1 mg/ml (unpublished observations). It certainly suggests that motor neuron death from envenomation, as reported by Lamb and Hunter (1904), is not caused by CTX.

Most likely motor neuron death was attributable to the presynaptic neurotoxins such as beta-bungarotoxin or nigexine. Experimentally induced programmed death of motoneurons can be achieved by in-ovo injection of the neurotoxin beta-bungarotoxin. Intramuscular administration of the snake toxin beta-bungarotoxin produces massive death of both lateral motor column motoneurons and doral root ganglion (DRG) neurons, resulting in a substantial increase in the number of pyknotic Schwann cells in both ventral and dorsal nerve roots. Haast claims to have treated ALS patients successfully with his neurotoxin formulation though it should be contra-indicated in this situation.

Muscular Dystrophy

Duchenne muscular dystrophy results from the lack of dystrophin, a cytoskeletal protein associated with the inner surface membrane, in skeletal muscle. The cellular mechanisms responsible for the progressive skeletal muscle degeneration that characterizes the disease are still debated. One hypothesis suggests that the resting sarcolemmal permeability for Ca(2+) is increased in dystrophic muscle, leading to Ca(2+) accumulation in the cytosol and eventually to protein degradation. Recent evidence suggests that cellular sodium regulation may also be abnormal in muscular dystrophy.

The effects of alpha-bungarotoxin pretreatment on calcium leakage activity (CLA) and AchR activity in MDX myotubes (from the mouse muscular dystrophy model) was studied (Carlson, “Effect of Alpha-bungarotoxin pretreatment on Calcium Leakage Activity (CLA) and ACHR activity in cultured MDX myotubes”, Abstracts, Society for Neuroscience, 29^{th} Annual Meeting, Oct. 1999, 735.14). Spontaneous transitions in the occurrence of CLA and AchR activity in individual patches from cultured mdx myotubes and results indicating that MDX patches exhibiting 100% CLA can be induced to exhibit AchR activity by the acquisition of an inside-out patch have led to the suggestions that AchRs contribute to CLA in dystrophic preparations. In order to further examine this hypothesis cultured MDX myotubes were exposed to 5 mcg/ml alpha-bungarotoxin for a period of 24 to 72 hours prior to recording single channel activity in the presence of 5×10⁷ M Ach (no alpha-toxin present). Examinations of two alpha-neurotoxin treated patches indicated reduced AchR and CLA in comparison to an untreated patch which exhibited a spontaneous increase in CLA (to an average of about 65 events per sec) at membrane potentials of 0 and 75 mV hyperpolarized from resting potential. These results suggested a reduction was consistent with the notion that AchRs contribute to CLA in MDX myotubes.

To determine whether the lack of dystrophin alters the occurrence of CLA and acetylcholine receptor (AChR) activity, the frequency of each event class was determined from several cell attached patches on non-dystrophic and dystrophic (mdx) myotubes. The frequency of CLA observed in the presence of ACh was significantly (P<0.05) elevated in mdx myotubes, an effect which was partly due to a significant (P<0.05) increase in the proportion of cell attached patches that exhibited 100% CLA with no AChR activity. Areas of MDX and nondystrophic membrane that exhibited reduced or absent AChR activity had significantly (P<0.01) and substantially elevated calcium leakage event frequencies. This inverse and discontinuous relationship between CLA and AChR activity provides further evidence that some CLA in dystrophic muscle is produced by clusters of AChRs that form unusual physical associations with the dystrophic cytoskeleton during the processes associated with receptor localization and stabilization. The information suggests that the administration of modified alpha-neurotoxin as a modulator of the nAchR would alleviate some of the symptoms of this disease.

Activity in Autoimmune Diseases

Myasthenia Gravis comes from the Greek and Latin words meaning grave muscular weakness. The most common form of MG is a chronic autoimmune neuromuscular disorder that is characterized by fluctuating weakness of the voluntary muscle groups. MG may affect any muscle that is under voluntary control. Certain muscles are more frequently involved and these include the ones that control eye movements, eyelids, chewing, swallowing, coughing and facial expression. Muscles that control breathing and movements of the arms and legs may also be affected. Weakness of the muscles needed for breathing may cause shortness of breath, difficulty taking a deep breath and coughing. The muscle weakness of MG increases with continued activity and improves after periods of rest. The muscles involved may vary greatly from one patient to the next. Weakness may be limited to the muscles controlling eye movements and the eyelids. This form of myasthenia is referred to as Ocular MG. In its severest form, MG involves many of the voluntary muscles of the body including those needed for breathing. The degree and distribution of muscle weakness for many patients falls in between these two extremes. When the weakness is severe and involves breathing, hospitalization is usually necessary.

MG is an autoimmune disease. Acetylcholine travels across the space to the muscle fiber side of the neuromuscular junction where it attaches to many receptor sites. In MG, there is as much as an 80% reduction in the number of these receptor sites. The reduction in the number of receptor sites is caused by an antibody that destroys or blocks the receptor site. Antibodies are proteins that play an important role in the immune system. For reasons not well understood, the immune system of the person with MG makes antibodies against the receptor sites of the neuromuscular junction. Abnormal antibodies can be measured in the blood of many people with MG. The antibodies destroy the receptor sites more rapidly than the body can replace them. Muscle weakness occurs when acetylcholine cannot activate enough receptor sites at the neuromuscular junction.

A number of tests may be used to establish a diagnosis of MG. A blood test for the abnormal antibodies can be performed to see if they are present. Electromyography (EMG) studies can provide support for the diagnosis of MG when characteristic patterns are present. The Edrophonium Chloride (Tensilon®) test is performed by injecting this chemical into a vein. Improvement of strength, immediately after the injection, provides strong support for the diagnosis of MG. Sometimes all of these tests are negative or equivocal in someone whose story and examination still seem to point to a diagnosis of MG. The positive clinical findings should probably take precedence over negative confirmatory tests.

There is no known cure for MG, but there are effective treatments that allow many, but not all people with MG, to lead full lives. Common treatments include medications, thymectomy and plasmapheresis. Spontaneous improvement, even remission, may occur without specific therapy. Medications are most frequently used in treatment. Anticholinesterase agents allow acetylcholine to remain at the neuromuscular junction longer than usual so that more receptor sites can be activated. Corticosteroids and immunosuppressant agents may be used to suppress the abnormal action of the immune system that occurs in MG. Intravenous immunoglobulins (IVIg) are sometimes used to affect the function or production of the abnormal antibodies also. Thymectomy (surgical removal of the thymus gland) is another treatment used in some patients. Thymectomy frequently lessens the severity of the MG weakness after some months. In some people, the weakness may completely disappear. This is called a remission. The degree to which the thymectomy helps varies with each patient. Plasmapheresis or plasma exchange may be useful in the treatment of MG also. This procedure removes the abnormal antibodies from the plasma of the blood. The improvement in muscle strength may be striking but is usually short-lived since production of the abnormal antibodies continues. When plasmapheresis is used, it may require repeated exchanges. Plasma exchange may be especially useful during severe MG weakness or prior to surgery. Treatment decisions are based on knowledge of the natural history of MG in each patient and the predicted response to a specific form of therapy. Treatment goals are individualized according to the severity of the MG weakness, the patient's age and sex, and the degree of impairment.

A lot of attention in MG research has focused on the acetylcholine receptor epitopes and antibodies to them. Some attention has also been focused on those components that may trigger the production of these antibodies. Brenner et al. (1989) showed that the stimulation of peripheral blood lymphocytes with the Epstein-Barr virus (EBV) from most patients with MG caused the production of antibodies to the acetylcholine receptor. The in-vitro synthesis of anti-acetylcholine receptor antibodies was found to positively correlate with both the patients' sera antibody titers and with the severity of disease. Yourist et al. (1983) reported the inhibition of HSV-1 by modified cobratoxin in tissue culture and the protection of mice following intracranial injection of the virus. Vargas and Cortes (1995) treated 78 individuals suffering from HSV-1, HSV-2 and VZV with modified cobratoxin.

Another interesting observation made by Duggan et al. (1988) was in patients with Mycobacterium leprae. When peripheral blood lymphocytes were hybridized with a lymphoblastoid line some of the antibodies produced cross-reacted with the acetylcholine receptor. The antibody was able to inhibit the binding of alpha-bungarotoxin to the acetylcholine receptor and could be blocked by ssDNA. Anti-idiotype antibodies containing the acetylcholine receptor domain recognized these antibodies also. The antibodies were found to share idiotypes to those found in patients with myasthenia gravis though the patients with M. leprae showed no signs of MG. It was reported that there are anti-acetylcholine receptor antibodies that bind to proteins in gram negative bacteria.

The effects of immunizing with a monoclonal antibody (mAb) that recognizes all long-chain curaremimetic toxins (Pillet et al., 1992) have been studied. The mAb binds to toxin residues that make contact with the toxin's target, e.g., the nicotinic acetylcholine receptor and also recognizes (−) nicotine, an agonist of this receptor. Injection in rabbits of the mAb (MST2) mixed with adjuvant, elicited anti-idiotypic (anti-Id) antibodies that inhibited binding of the toxin to the acetylcholine receptor. A proportion of these anti-Id antibodies specifically bound to the acetylcholine receptor and thereby mimicked the toxin. Furthermore, rabbits immunized with MST2 elicited auto-anti-anti-Id antibodies capable of binding the neurotoxin. Similar observations have been made by the applicants where antibodies to the torpedo receptor and antibodies to alpha-cobratoxin appeared to interact in ELISA studies. Patients injected with the modified cobratoxin of the invention develop high titers to the protein.

In antibody binding studies, a peptide from the alpha subunit (388-408) of the bound antibodies raised against free AChR or against membrane-bound AChR. This peptide also bound specifically both ¹²⁵I-labelled bungarotoxin and cobratoxin, while other peptides had no binding activity (Atassi et al, 1988). The majority of antibodies from MG bind to segment 371-378 on the acetylcholine receptor alpha-subunit (Marx et al., 1989) that also binds bungaro —and cobratoxin. These findings did not encompass all MG patients tested and leads to speculation about differing forms of MG resulting from the varying specificities of the auto-antibodies produced.

It is proposed that the administration of alpha-neurotoxins to people with MG may have 2 mechanisms of value; the first permits modified neurotoxins to compete for binding to the AchR with the host antibodies, secondly, the production of antibodies to alpha-neurotoxins could neutralize the autoimmune antibodies—in a sense vaccinating the host against the disease and has been proposed for protection against Rabies.

The conversion of neurotoxins with hydrogen peroxide is relatively simple and can be achieved at relatively high protein concentrations (10 mg/ml). The reactive species in cheap and abundant. The process employed by Sanders above required the addition of some agents which preferably required removal post reaction. Agents such as catalase, copper sulphate and phosphate buffers. While these agents have proven safe in chronic toxicity tests it is always desirable to reduce the number of chemicals where possible to minimize their effects on the host.

The reaction procedure with hydrogen peroxide occurs over the course of 7-14 days and loss of toxicity occurs within that time period. Miller's studies (1977) have shown that with continued oxidation, the loss of the tryptophan residue can be observed. This coincides with the method for following the reaction of neurotoxins with ozone (Chang et al, 1990, Mundschenk Pat. No. 5,989,857). Studies conducted by Miller suggest that the loss of toxicity is due mainly to the reduction in the number of disulphide bonds.

Alpha-neurotoxin solution, i.e. cobratoxin, is filter sterilized to remove bacteria. It can be dissolved in saline and made up to final volume minus H₂O₂ volume (see Sanders, Pat. No. 3,888,977). H₂O₂ should be added last while agitating. Final protein product concentration is 10 mg/ml. Conceivably the protein level can be increased concomitant with an increase in the level of H₂O₂ to yield 20 or 30 mg/ml solutions. There is a 1000 fold molar excess of H₂O₂ relative to neurotoxin. This would increase production while keeping the handling volume to a minimum. The solution needs to be diluted prior to filling and administration (e.g. to 500 mcg/ml). Any suitable preservative for parenteral administration can be employed such as methyl paraben, benzalkonium chloride or metacreosol. For oral administration of the neurotoxin the-modified protein must be combined with benzalkonium chloride at a protein:detergent ratio of between 1:6 to 1:8, and preferably 1:7.5 for solutions with modified cobratoxin.

As noted in the summary of the invention above, there are provided alternative methods of drug production. Other oxidizing and reduction techniques produce modified neurotoxins with antiviral activity (Miller et al, 1977). In this application a method employing heat treatment of cobratoxin and venom is disclosed. These novels methods of production give the option of generating proteins with subtle differences that have great importance to their application. Excessive exposure to heat is a mechanism that can be employed to investigate stability and heat-stress studies are commonly employed to assess the heat sensitivity of a protein and to simulate the passage of time. It can also be employed as a method to “denature” proteins for application as vaccines. It was through these studies that this invention was made.

The most interesting aspect of heat modification is the discovery, unlike H₂O₂ modified material, of the failure of this preparation to demonstrate antiviral activity in assays with rabies virus where the cell lines are devoid of nAchRs. In this aspect the preparation was similar to formaldehyde treated venom. However, the autoclaved (heat denatured) material retains the ability to bind to and compete with native cobratoxin for binding to the nAchR. This fact underscores the subtle differences between these difference forms of protein modification and emphases the role of the activity on nAchR for the field of the invention.

The neurotoxin's resistance to high temperature also permits the use of heat as a modification to the original formula developed by Sanders. The instability of hydrogen peroxide to heat permits the use of elevated temperatures as a method to drive off excess hydrogen peroxide when the reaction with venom or purified neurotoxins is deemed complete, possibly in a situation where catalase is unobtainable. However unless gentle heat is employed or the solution is diluted to 1 mg/ml or less the use of high temperature should be avoided. Lower temperature elevations are advised in solutions containing proteins concentrations greater than 1 mg/ml.

While the invention has been described, and disclosed in various terms or certain embodiments or modifications which it has assumed in practice, the scope of the invention is not intended to be, nor should it be deemed to be, limited thereby and such other modifications or embodiments as may be suggested by the teachings herein are particularly reserved especially as they fall within the breadth and scope of the appended claims.

The cloning of a variety of neurotoxins have proven successful though the majority of efforts have focused upon those toxins which are found only in low quantities in native venoms (Fiordalisi et al., (1996) Toxicon 34, 2, 213-224, Krajewski et al (1999) “Recombinant m1-toxin” presented at the 29^{th} Annual Meeting of the Society for Neuroscience) and also with the desire to produce mutants to study structure/function relationships (Smith et al., (1997) Biochemistry, 36, no. 25, 7690-7996. Cobratoxin has been cloned (Antil S, Servent D and Menez A. J Biol Chem (1999) Dec. 3;274(49):34851-8) though it is abundant and easily obtained from natural sources in order to study the effect of mutations on its interactions with the acetylcholine receptor. Several bioengineered variants have been proposed by the author who was a contributor to the Smith et al. (1997) paper which replace the residues required for disulphide bond formation with other residues so as to closely mimic the effects of chemical or heat modifications. This substitution is obvious because the heat modified protein migrates in sizing gels are if it were exposed to b-mercaptoethanol, a reducing agent that cleaves disulphide bonds. As these amino acid substitutions must be expressed in-vivo the availability of modifications are limited to the use of native residues (the standard 20 naturally occurring amino acids) and the host to be employed for expression. In the host the codon usage will be important in ensuring efficient and maximal expression of the novel protein. Theoretically any amino acid can be substituted for cysteine but as this is a more costly approach to generating cobratoxin variants relative to synthetic peptide techniques certain residues have been selected which best reproduce the protein characteristics resulting from chemical exposure. It is usual in this circumstance to make what are considered to be conservative substitutions. As a result, it has been chosen to initially limit the cysteine replacement to the following residues; methionine (M), glutamic acid (E), aspartic acid (D), glutamine (Q), asparagine (N), serine (S), glycine (G) and alanine (A). Methionine incorporation would could be considered to be the more conservative substitution by replacing one sulphur-containing residue for another. Unlike cysteine, methionine cannot form disulphide bonds. Methionine also reacts readily with oxidizing agents to produce the sulfone derivative therefore the purified product can be exposed to chemical agents to confer upon the protein other desirable properties (i.e. low immunogenicity). Also the presence of methionine also allows for the cleavage of the protein into fragments employing cyanogen bromide. Cleavage of the native cobratoxin and modified protein is easily achieved with serine proteases (i.e., trypsin) but at sites containing positive residues. This permits also the evaluation and production of smaller peptide fragments for biological activity (Hinmann et al., 1999). The conversion of cysteine to cysteic acid by oxidation also argues for the substitution by other acidic residues such as E, D, Q, N and S. The substitution of E and D for cysteine is estimated to produce a protein with a pI similar to that of modified cobratoxin (pI=4.5). The substitution of cysteine with the residues glycine and alanine would represent standard “neutral” substitutions. The method for creating these genes has been described previously (Smith et al., 1997). The codon usage of the DNA fragments is optimized for use in commercially used bacterial and yeast expression systems Escherichia coli and Pichia pastoris respectively.

Current technology has also allowed for the production neurotoxins through peptide synthesis. Many smaller neurotoxins (from conus snails, bee venom and scorpion venom) are routinely produced by synthetic peptide methodology (Hopkins et al., (1995) J. Biol. Chem., 270, no. 38, 22361-22367, Ashcom and Stiles, (1997) Biochem. J. 328, 245-250, Granier et al., (1978) Eur. J. Biochem, 82, 293-299 and Sabatier et al., (1994) Int. J. Pept. Protein Res., 43, 486-495) and some are available from commercial organizations. The above references also describe the synthesis of such peptides incorporating mutant residues (Hopkins et al. (1995) and Sabatier et al (1994)). Current techniques in peptide chemistry allow for proteins in excess of 80 amino acids can be reliably produced using automated Fmoc solid phase synthesis (ABI 433A Peptide Synthesizer, Perkin Elmer—see www.perkin-elmer.com). Non-native amino acids (acetamidomethyl cysteine, carboxyamidomethyl cysteine, cysteic acid, kynurenine and methionine sulphone) can be acquired from Advanced Chemtech (Louisville, Ky.) or Quchem (Belfast, Ireland). Other oxidized or alkylated amino acid variants are available from these agents. The generation of a synthetic version of the neurotoxin can be achieved by substituting primarily the cysteine residues (from 1 pair to all 5 disulphide couples) with those residues described above to mimic the effects of the various chemical modifications. Furthermore the substitution of other native and non-native residues for cysteine can be investigated in an attempt to identify neurotoxin variants with improved biological activity. Also peptide fragments from within the cobratoxin sequence can be created (analogous to Hinmann et al., (1999), Immunoparmacol. Immunotoxicol, 21 (3), 483-506) and examined for receptor binding activity.

As there are several drug preparation techniques, some described in detail above, it is submitted that they would be essentially the same with respect to nAchR binding under the Code of Federal Regulations Title 21, Volume 5, Part 310, Section 310.6, b (1) which states that identical, related, or similar drugs includes other brands, potencies, dosage forms, salts, and esters of the same drug moiety as well as of any drug moiety related in chemical structure or known pharmacological properties.

The normal dosage of the present modified neurotoxin for the average adult is approximately 0.3 mg per day. The dosages are correspondingly adjusted for younger or older patients of greater or less body weight. The maximum dosage need not exceed 1 mg per day. Dosages of 0.03 mg have been found to be effective though with slower onset of relief. While a patient may be given the modified neurotoxin as infrequently as every other week, it is preferred that the composition be administered at least weekly, and preferably every other day or daily. The composition may be administered orally, subcutaneously, intramuscularly or intravenously. Parenterally, either subcutaneous or intramuscular injection is preferred. While the correct formulation with benzalkonium chloride will permit oral administration through absorption through the oral mucosa (preferably sublingually), this formulation may also permit administration otically. Furthermore transdermal delivery may be affected if formulated in an appropriate cream or lotion base using benzalkonium chloride as a permeation enhancer.

### EXAMPLE 1

### nAchR Binding Activity

Natural cobra alpha-neurotoxin is toxic because of its' high affinity binding to acetylcholine receptors (ACHR). High temperature and oxidation of cobra alpha-neurotoxin abolishes the toxicity of the alpha neurotoxin, as determined by the absence of lethality by IP or IM injection of modified cobratoxin into mice. Binding of modified cobratoxin into NAchR in vitro has been determined to still occur though with greatly decreased affinity. Modified cobratoxin-ACHR binding in vitro is determined by a modification of an enzyme immunoassay (EIA) developed by B. G. Stiles (1991) for the detection of postsynaptic neurotoxins.

In this assay, neurotoxin or oxidized neurotoxin is bound by hydrophobic interaction to the wells of a polystyrene immunoassay plate. After washing of the wells, whole acetylcholine receptor (ACHR) from Torpedo californica isolated by the method of Froehner and Rafto (1979) is placed in the wells and binds to polystyrene bound neurotoxin or oxidized neurotoxin. Bound ACHR is then detected by ACHR specific antibody. The specificity of binding of ACHR to polystyrene bound Modified cobratoxin has been determined by inhibition of binding by carbamylcholine chloride and by native cobratoxin.

Based first upon the natural high affinity binding of un-modified cobratoxin to ACHR and also upon our determination of the continued ability of oxidized cobratoxin to bind to ACHR, though with greatly reduced affinity, the activity of Modified cobratoxin in vivo is assumed to occur at the level of acetylcholine receptors or acetylcholine-like receptors. The binding of modified cobratoxin with eel ACHR in vitro forms the basis for the potency assay for these drugs.

Briefly, the Modified cobratoxin potency assay is performed as follows. Test modified cobratoxin controls based upon therapeutic activity (high activity, low activity and no activity) as well as BSA, as a reagent control, at a concentration of 10 ug/ml carbonate buffer are each exposed to four replicate wells of an EIA plate overnight at room temperature. After washing of the wells with phosphate buffered saline containing 0.05% Tween-20 (PBST), the wells are blocked with PBS SuperBlock (PBSSB; Pierce; Rockford, Ill.) according to the manufacturers directions. Eel ACHR at a concentration of 10 ug/ml PBSSB containing 0.05% Tween-20 (PBSSB0.05T) is placed in all wells and incubated at room temperature for 2 hours. After washing of the wells with PBST, mouse monoclonal antibody specific for ACHR is placed in all wells and incubated for 1 hour at room temperature. ACHR bound monoclonal antibody is identified by anti mouse IgG-biotin (Jackson ImmunoResearch; West Grove, Pa.) and streptavidin-HRP (SAHRP) (Pierce). Color development is generated by TMB (2-part; Kirkegaard & Perry; Gaithersburg, Md.) and stopped by the addition of 1M phosphoric acid. Absorbance is determined at 450 nm. The average absorbance due to the BSA reagent control wells (with an A₄₅₀ of 0.070 or less) is subtracted from all other average absorbance levels generated by test and control Modified neurotoxin. Test Modified cobratoxin absorbance is divided by the average absorbance due to the high therapeutic activity control and multiplied by 100 to produce the percent potency of the test modified neurotoxin.

### EXAMPLE 2

### Heat Modification Procedure

Cobratoxin (CT) was dissolved in distilled water or physiological saline (0.9%) is autoclaved (121° C., 20 minutes). The solution concentrations ranged from 100 mcg/ml to 900 mcg/ml. Following this exposure the container and solution remained intact and clear though with some precipitation. At lower concentrations very little precipitation was observed and there were no obvious indications of deterioration. When measured, the protein concentration did not change significantly even when the level of precipitation appeared excessive. When examined by PAGE the autoclaved CT migrated similar to being in a reduced state. The intensity of the staining was reduced though the same quantity of protein was loaded for each pair suggesting an event like oxidation was responsible for the effects observed. There was no discernible difference in the resulting product when autoclaving was conducted in distilled water or saline for injection. The presence of a preservative did not appear to alter the appearance of the autoclaved protein when analyzed by PAGE. This study suggests that CT maintains an overall molecular weight of circa 8,000d following autoclaving though some smaller fragments can be observed below 8,000d. Additionally UV analyses of the autoclaved samples indicate there are no observable changes in the absorption characteristics, the tryptophan residue remaining intact which suggests that this was a milder form of oxidation that hydrogen peroxide (Miller et al, 1977) or ozone (Chang et al., 1990).

CT was convenient to employ for these studies because potency and toxicity are interwoven. The injection of autoclaved cobratoxin (600 mcg/ml, 0.01% BC) into 4 mice (sc, 50 mcl-30 mcg) produced no toxic indications and no deaths over 3 days of observations. Injection of the control, non-autoclaved cobratoxin (600 mcg/ml, 0.01% BC) into 4 mice (sc, 50 mcl-30 mcg) resulted in deaths averaging 20.5 minutes. The injection of solutions autoclaved at 100, 300 and 900 mcg/ml also failed to kill mice.

### EXAMPLE 3

### Inhibition of Rabies

-A second example includes the inhibition of rabies virus, which is being studied at the time of this writing. It will be furnished as a part of a later filing.

### EXAMPLE 4

### Induction of Cobratoxin Antibodies

The administration of modified cobratoxin elicits an immune response which can be monitored in humans over the period of a standard immunization protocol (3 months). In humans, polyclonal antibodies can be induced by daiily injections of 100 mcg/ml solutions of modified cobratoxin with the appearance of antibodies within 2 weeks. EIA determined titers have been recorded in some individuals greater than 100,000. The antibody elicited cross-reacts with native alpha-cobratoxin through ELISA analysis and it is known that this antibody would not be protective against parenteral administration of the native protein (cobratoxin) under a standard vaccination protocol. Such an immune response to modified cobratoxin does not adversely affect the efficacy of the drug as demonstrated by modified venom and cobratoxin treatment of patients with neurological disorders some for periods up to 12 years. It has been found that high concentration bolus doses of modified cobratoxin (>1 mg/ml) can induce injection site reactions in naive patients. This reaction has been characterized as a Jones-Molt reaction. This results in naive patient with drug product that is highly aggregated. The immune response to the oral administration of modified cobratoxin is results in a much reduced titer than that observed for the parenteral format. Additionally, upon switching from parenteral to oral formulations a reduction in antibody titer is recorded.

Rabbit polyclonal antibodies and mouse monoclonal antibodies have been generated using modified cobratoxin. The rabbit polyclonal antibodies were induced by injecting 20 mcg with Freund's complete adjuvant following stardard protocols to the industry. The monoclonal antibodies were generated by injecting 30 mcg of alum precipitated modified cobratoxin i.p. on Day 0. On Day 30, 60 mcg of modified cobratoxin (without alum) plus 50 mcg of “Poly A Poly U” (Sigma). On Day 44, 20 mcg of modified cobratoxin (without alum) plus 50 mcg of “Poly A Poly U”. On Day 58, of modified cobratoxin (without alum) plus 50 mcg of “Poly A Poly U” and 3 day later mouse spleen was fused with immortalized cells (NS1 cell line) followed standard practices. Each antibody type recognize both modified and native cobratoxin. Furthermore, ELISA studies have shown that these antitoxin antibodies cross react with antibodies against the nAchR by blocking the anti-nAchR antibody binding to the target, an attribute desirable in patients with MG. These observations suggest that a high antibody response, even in the absence of an adjuvant, can be selectively induced using injectable formats where a high circulating antitoxin titer may be desirable such as in the condition MG.

The applicants' experiences in several disorders (Multiple Sclerosis, Amyotrophic Lateral Sclerosis, Adrenomyeloneuropathy and Ataxias) demonstrate improved function (muscle strength, walking speed) and endurance, symptoms which are prevalent also in MG. The mechanism is assumed to involve mainly presynaptic acetylcholine receptors. Haast (1982) reports that patients receiving native neurotoxin combinations reported similar effects. While cobratoxin does bind to the muscle receptor in-vitro very little or no paralysis is observed in mice injected with the toxin which supports the above theory.

### EXAMPLE 5

### Human Subject with ALS

A human volunteer with confirmed ALS was administered both oxidized and autoclaved alpha-cobratoxin in an oral formulations comprising 600 mcg/ml of the neurotoxin and 0.01% Benzalkonium chloride suspended in 0.9% physiological saline. In the absence of anticholinergic therapy the patient reported stiffness and pain upon rising and leg pain during the day. This combined with reduced endurance and strength comprised the symptoms to be followed when assessing the new formulation. Following an overnight abstinence from other anticholinergic drugs, he administered 1 spray sublingually (equivalent to 0.1 ml volume). He noted improved pain and strength approximately 15 minutes post administration. Administration of either solution throughout the day provided satisfactory improvements in strength, endurance and relief from pain equivalent to prior therapeutic modalities. These observations confirm the importance of the nAchR binding properties of both formulations. The patient has employed oral and injectable formulation s of the modified neurotoxin for over 3 years. Electromyograph recordings have indicated that the rate of deterioration associated with the disease has reduced significantly.

### EXAMPLE 6

### Human Subject with MS

A human volunteer with confirmed MS was administered oxidized alpha-cobratoxin in an oral formulation comprising 500 mcg/ml of the neurotoxin and 0.007% Benzalkonium chloride suspended in 0.9% physiological saline. In the absence of anticholinergic therapy the patient reported stiffness and pain upon rising and leg pain during the day. This combined with reduced endurance and strength comprised the symptoms to be followed when assessing the new formulation. Following an overnight abstinence from other anticholinergic drugs, he administered 1 spray sublingually (equivalent to 0.1 ml volume). He noted improved pain and strength approximately 15 minutes post administration. Administration of the solution throughout the day provided satisfactory improvements in strength, endurance and relief from pain equivalent to prior therapeutic modalities. Following 3 years of use, the patient continues to employ this product and reports his disease has stabilized and the rates of deterioration has significantly declined.

### EXAMPLE 7

### Human subject with MS

A human volunteer with confirmed MS was administered oxidized alpha-cobratoxin in a parenteral formulation comprising 500 mcg/ml of the neurotoxin and 0.001% Benzalkonium chloride suspended in 0.9% physiological saline. In the absence of anticholinergic therapy the patient reported stiffness and pain upon rising and leg pain during the day. This combined with reduced endurance and strength comprised the symptoms to be followed when assessing the new formulation. Following an overnight abstinence from other anticholinergic drugs, she administered 1 injection (equivalent to 0.5 ml volume). She noted improved pain and strength approximately 20 minutes post administration. Administration of the solution throughout the day provided satisfactory improvements in strength, endurance and relief from pain equivalent to prior therapeutic modalities. Following 3 years of use, the patient continues to employ this product and reports her disease has stabilized.

### EXAMPLE 8

### Human Subject with Adrenomyeloneuropathy (AMN)

A human volunteer with confirmed AMN was administered oxidized alpha-cobratoxin in an injectable formulation comprising 600 mcg/ml of the neurotoxin and 0.01% Benzalkonium chloride suspended in 0.9% physiological saline. In the absence of anticholinergic therapy the patient reported reduced strength and poor endurance. This combined with reduced endurance and strength comprised the symptoms to be followed when assessing new formulations. Administration of the solution (0.2 cc t.i.d.) throughout the day provided satisfactory improvements in strength and endurance. Measured conduction velocities were recorded as improved over scores recorded prior to the initiation of therapy. This data strongly indicates the drug(s) are modulating the signals generated by the nerve cells and most reasonably through their interaction with nAchRs. The patient continues to employ this product and reports disease stabilization with treatment over 2 years.

While the invention has been described, and disclosed in various terms or certain embodiments or modifications which it has assumed in practice, the scope of the invention is not intended to be, nor should it be deemed to be, limited thereby and such other modifications or embodiments as may be suggested by the teachings herein are particularly reserved especially as they fall within the breadth and scope of the appended claims.

What is claimed is:

1. A method of treatment of animals suffering from neurological disorders comprising administering to the animal a disease mitigating dosage of a detoxified and neurotropically active modified alpha-neurotoxin composition which targets nicotinic acetylcholine receptors.

2. The method of claim 1 wherein the detoxified and neurotropically active modified composition comprises a fraction containing the alpha-neurotoxins.

3. The method of claim 1 wherein the alpha-neurotoxins are selected from the group consisting of alpha-bungarotoxin, kappa-bungarotoxin, alpha-cobratoxin, alpha-cobrotoxin, alpha-conotoxins (G1, M1, S1, S1A, ImI), alpha-dendrotoxin and erabutoxin.

4. The method of claim 1 wherein the alpha-neurotoxin composition comprises alpha-cobratoxin.

5. The method of claim 1 where in humans the dosage of the composition is from about 0.05 to 10 ml based on a 0.1% solution of the modified cobratoxin per 150 lbs body weight.

6. The method of claim 5 wherein the dosage is from 0.4 to 3 ml.

7. The method of claim 5 wherein the dosage is administered in a frequency of from every other week to daily.

8. The method of claim 5 wherein the dosage is administered at least weekly.

9. The method of claim 5 wherein the dosage is administered at least daily.

10. The method of claim 5 wherein composition administration methods include by injection (subcutaneous, intramuscular and intravenous), orally, otically and by intradermal routes.

11. The method of claim 1 wherein subject neurological condition benefits from improved nerve conduction and modulation.

12. The method of claim 11 wherein the neurological condition is selected from the group comprising Amyotrophic Lateral Sclerosis, other spinal atrophies, Multiple Sclerosis, Myasthenia Gravis, Muscular Dystrophy, Leukodystrophies, Adrenomyeloneuropathy and Ataxias.

13. A method of vaccinating a subject comprising administering to the subject a immunogenic amount of a detoxified and neurotropically active modified neurotoxin composition with or without the inclusion of an adjuvent.

14. The method of claim 13 wherein composition administration methods include by injection (subcutaneous, intramuscular and intravenous), orally, otically and by intradermal routes.

15. The method of claim 13 wherein blocking neurotropic viruses that employ the nAchR for cell entry comprises administering to a subject an amount of a detoxified and neurotropically active modified neurotoxin composition.

16. The method of claim 15 wherein composition administration methods include by injection (subcutaneous, intramuscular and intravenous), orally, otically and by intradermal routes.

17. A composition comprising an administrable form of a detoxified and neurotropically active modified snake venom neurotoxin wherein a Naja venom neurotoxin is alpha-cobratoxin and the composition is atoxic.

18. The composition of claim 17, wherein the alpha-cobratoxin can be administered orally when combined in a solution with benzalkonium chloride.

19. The composition according to claim 18, wherein the alpha-cobratoxin can be administered orally when combined in a solution with benzalkonium chloride at a protein: detergent ratio of between 1:6 to 1:8, and preferably 1:7.5.
